(19) United States
(12) Patent Application Publication
(10) Pub. No.: US 2007/0141053 A1
(43) Pub. Date: Jun. 21, 2007

(54) Treatment of inflammatory respiratory diseases

(75) Inventors: Lars Breimer, Chatham, NJ (US); Reinhard Von Roemeling, Ridgefield, CT (US)

Correspondence Address: MILLEN, WHITE, ZELANO AND BRANIGAN, P.C.;C/O BERLEX BIOSCIENCES, 2200 CLARENDON BOUEVARD, SUITE 1400, ARLINGTON, VA, 22201, US

(73) Assignee: Schering Aktiengesellschaft, Berlin (DE)

(21) Appl. No.: 10/555,577

(22) PCT Filed: May 7, 2004
(86) PCT No.: PCT/US04/14249

§ 371 Date: Nov. 27, 2006

Related U.S. Application Data
(60) Provisional application No. 60/468,976, filed on May 9, 2003.

(51) Int. Cl. A61K 39/395

(52) U.S. Cl. 424/144.1

(57) ABSTRACT

The present invention relates to the use of immune modulatory factors which act at CD114, CD116, and or CDw131 to successfully treat various forms of inflammatory respiratory disease, including, but not limited to ARDS, IRDS, SARS, PRRS, PEARS and SIRS.

This application claims the benefit of U.S. Provisional Application Ser. No. 60/468,976, filed May 9, 2003, which is incorporated herein in full by reference.

### BACKGROUND OF THE INVENTION

Respiratory syndromes comprise a number of disease states with different etiologies. Examples are severe acute respiratory syndrome (SARS), acute (adult) respiratory syndrome (ARDS), and infant respiratory syndrome (IRDS). In animals, similar diseases have been observed. For example in swine, porcine reproductive and respiratory syndrome (PRRS), swine infertility and respiratory syndrome (SIRS), and porcine epidemic abortion and respiratory syndrome (PEARS) have been described that cause significant losses in pig breeding farms.

The purpose of the present invention is to provide a treatment for a number of respiratory diseases, which are currently either without any treatment or for which the presently available treatments are only useful to a minor extent.

Severe acute respiratory syndrome (SARS) is a disease that has been described in patients in a number of countries in Asia, in USA, and in Europe. SARS has been associated etiologically with a novel coronavirus, SARS-CoV (Kziazek; N Engl J Med; Drosten, N Engl J Med).

The incubation period for SARS is typically 2-7 days; however, isolated reports have suggested an incubation period as long as 10 days (CDC Report, Mar. 28, 2003). The illness begins generally with a prodrome of fever (>38.0° C.). Fever often is high, sometimes is associated with chills and rigors, and might be accompanied by other symptoms, including headache, malaise, and myalgia. At the onset of illness, some persons have mild respiratory symptoms. Typically, rash and neurologic or gastrointestinal findings are absent; however, some patients have reported diarrhea during the febrile prodrome.

After 3-7 days, a lower respiratory phase begins with the onset of a dry, nonproductive cough or dyspnea, which might be accompanied by or progress to hypoxemia. In 10%-20% of cases, the respiratory illness is severe enough to require intubation and mechanical ventilation. The case-fatality rate among persons with illness meeting the current WHO case definition of SARS is approximately 3%. However, the latest WHO report on death rates in SARS refer to approx. 50% In patients of 60 years or older and to an overall rate of 13-15%.

Chest radiographs might be normal during the febrile prodrome and throughout the course of illness. However, in a substantial proportion of patients, the respiratory phase is characterized by early focal interstitial infiltrates progressing to more generalized, patchy, interstitial infiltrates. Some chest radiographs from patients in the late stages of SARS also have shown areas of consolidation.

Early in the course of disease, the absolute lymphocyte count is often decreased. Overall white blood cell counts have generally been normal or decreased. At the peak of the respiratory illness, approximately 50% of patients have leukopenia and thrombocytopenia or low-normal platelet counts (50,000-150,000/μL). Early in the respiratory phase, elevated creatine phosphokinase levels (as high as 3,000 IU/L) and hepatic transaminases (two to six times the upper limits of normal) have been noted. In the majority of patients, renal function has remained normal.

The severity of illness might be highly variable, ranging from mild illness to death. Although a few close contacts of patients with SARS have developed a similar illness, the majority have remained well. Some close contacts have reported a mild, febrile illness without respiratory signs or symptoms, suggesting the illness might not always progress to the respiratory phase.

Treatment regimens have included several antibiotics to presumptively treat known bacterial agents of atypical pneumonia. In several locations, therapy also has included antiviral agents such as oseltamivir or ribavirin. Steroids have also been administered orally or intravenously to patients in combination with ribavirin and other antimicrobials. At present, the most efficacious treatment regimen, if any, is unknown. Thus there is a need in the art for effective method for treating SARS.

Adult (acute) respiratory distress syndrome is a respiratory failure caused by various acute pulmonary injuries and characterized by noncardiogenic pulmonary edema, respiratory distress, and hypoxemia. It is precipitated by various acute processes that directly or indirectly injure the lung, eg, sepsis, primary bacterial or viral pneumonias, aspiration of gastric contents, direct chest trauma, prolonged or profound shock, burns, fat embolism, near drowning, massive blood transfusion, cardiopulmonary bypass, O₂ toxicity, acute hemorrhagic pancreatitis, inhalation of smoke or other toxic gas, and ingestion of certain drugs (Merck Index).

The initial lung injury is poorly understood. Animal studies suggest that activated WBCs and platelets accumulate in capillaries, the interstitium, and airspaces; they may release prostaglandins, reactive oxygen species and free radicals of oxygen, proteolytic enzymes, and other mediators (such as tumor necrosis factor and interleukins), which injure cells, promote inflammation and fibrosis, and alter bronchomotor tone and vasoreactivity.

When the pulmonary capillary and alveolar epithelia are injured, plasma and blood leak into the interstitial and intra-alveolar spaces. Alveolar flooding and atelectasis result; atelectasis is due in part to reduced surfactant activity. The injury is not homogeneous and affects mainly the dependent lung zones. Within 2 to 3 days, interstitial and bronchoalveolar inflammation develops, and epithelial and interstitial cells proliferate. Then, collagen may accumulate rapidly, resulting in severe interstitial fibrosis within 2 to 3 wk. These pathologic changes lead to low lung compliance, decreased functional residual capacity, ventilation/perfusion imbalances, increased physiologic dead space, severe hypoxemia, and pulmonary hypertension.

Many approaches to the prevention and management of ARDS have been unsuccessful or inconclusive. Treatments that have not improved outcome or prevented ARDS include monoclonal antibody to endotoxin, monoclonal antibody to tumor necrosis factor, interleukin-1 receptor antagonist, prophylactic (early) PEEP, extracorporeal membrane oxygenation and extracorporeal CO₂ removal, IV albumin, volume expansion and cardiotonic drugs to increase systemic O₂ delivery, corticosteroids in early ARDS, parenteral ibuprofen to inhibit cyclooxygenase, prostaglandin E₁, and pentoxifylline.

Porcine Reproductive and Respiratory Syndrome (PRRS) is considered the most economically important viral disease of intensive swine farms in Europe and North America. The disease may also be referred to as Swine Infertility and Respiratory Syndrome (SIRS) by some veterinary and swine industry professionals.

Acute outbreaks of PRRS within a swine herd can cause some dramatic symptoms. In the breeding herd, sows may display an elevated body temperature, reduced appetite and lethargy. The European reports also indicate an increase in bruising and a blue ear appearance of white sows (Done, Misset-PIGS, 1995). Increases in the number of premature farrowings (abortions), stillbirths, mummified fetuses and weak piglets at birth are often reported. Agalactia may also occur among lactating sows. Stillbirths and mummies may increase to 35% and abortions can exceed 10% (Dee et al., Compendium of Continuing Education for Practicing Veterinarians, 1994).

An important feature associated with the PRRS virus is the immunosuppressive effect it has, particularly in piglets and weanling pigs. An affinity for PRRS virus of sow origin to infect swine alveolar monocytes has been demonstrated (Voicu et al., 1994) and the virus causes death of pulmonary alveolar macrophages (Hill, 1996). This feature is consistent with the high incidence of secondary pathogenic infections among suckling and nursery pigs. It appears that normal levels of bacterial agents may become pathogenic when pigs contract a PRRS virus infection.

In the USA only one PRRS vaccine is currently labeled for swine use. The product is a modified live virus vaccine, trade name RespPRRSr, manufactured by NobI Laboratories. The vaccine is only approved for use in pigs from 3 to 18 weeks of age. However, significant “off-label” use is being prescribed by swine veterinarians working with large herds experiencing PRRS cases. In prescribing off-label use, veterinarians are accepting some risk that the modified live virus may increase disease risk among some classes of pigs (McCaw, 1995).

There is still debate among veterinarians as to when it is safe and effective to vaccinate various classes of pigs. One concern is the potential for problems in developing fetuses when pregnant sows are vaccinated with the modified live virus during late pregnancy (after 50 days). The universal opinion among swine health practitioners is that indiscriminate use of the vaccine should be avoided and that use without other herd management strategies to control PRRS will not be effective. Accordingly, there is a strong demand for a treatment effective in porcine respiratory syndromes.

### SUMMARY OF THE INVENTION

It is an object of the invention to provide a method of treating respiratory syndromes in patients and animals.

This and other objects of the invention are provided by one or more of the embodiments described below.

In one embodiment a method is provided of treating SARS by administering an agonist of CD114 (Granulocyte Colony Stimulating Factor Receptor (G-CSFR)) to a patient with SARS.

In a related embodiment, this invention is directed to the use of an agonist of CD114 for the preparation of a pharmaceutical composition for treating inflammatory respiratory disease. In another embodiment of the invention, a method is provided of treating SARS in which an immune stimulatory amount of an agonist of CD114 (Granulocyte Colony Stimulating Factor Receptor (G-CSFR)) is administered to a patient with SARS.

In another embodiment of this invention, a method is provided of treating SARS by administering an agonist of agonist of CD116 (Granulocyte-Macrophage Colony Stimulating Factor Receptor) or CDw131 is administered to a patient with SARS. In a related embodiment, this invention is directed to the use of an agonist of CD116 or CDw131 for the preparation of a pharmaceutical composition for treating inflammatory respiratory disease. In another embodiment of the invention, a method is provided of treating SARS in which an immune stimulatory amount of an agonist of CD116 or CDw131 is administered to a patient with SARS.

In yet another embodiment of the invention a method is provided of treating ARDS and IRDS. An agonist of CD114 (Granulocyte Colony Stimulating Factor Receptor (G-CSFR)) is administered to a patient with ARDS or IRDS. More specifically, an immune stimulatory amount of an agonist of CD114 is administered to a patient with ARDS or IRDS.

In still another embodiment of the invention, an agonist of CD116 (Granulocyte-Macrophage Colony Stimulating Factor Receptor) or CDw131 is administered to a patient with ARDS or IRDS. In a preferred embodiment, an immune stimulatory amount of an agonist of CD116 or CDw131 is administered to a patient with ARDS or IRDS.

In even another embodiment of the invention a method is provided of treating porcine reproductive and respiratory syndrome (PRRS). An agonist of CD114 (Granulocyte Colony Stimulating Factor Receptor (G-CSFR)) is administered to a swine with PRRS. More particularly, an immune stimulatory amount of an agonist of CD114 (Granulocyte Colony Stimulating Factor Receptor (G-CSFR)) is administered to a swine with PRRS.

In yet another embodiment of the invention another method is provided of treating PRRS. An agonist of CD116 (Granulocyte-Macrophage Colony Stimulating Factor Receptor) or CDw131 is administered to a swine with PRRS. More particularly, an immune stimulatory amount of an agonist of CD116 or CDw131 is administered to a swine with PRRS.

According to another aspect of the invention a method is provided of treating swine infertility and respiratory syndrome (SIRS). An agonist of CD114 (Granulocyte Colony Stimulating Factor Receptor (G-CSFR)) is administered to a swine with SIRS. More particularly, an immune stimulatory amount of an agonist of CD114 (Granulocyte Colony Stimulating Factor Receptor (G-CSFR)) is administered to a swine with SIRS.

According to another aspect of the invention a method is provided of treating SIRS. An agonist of CD116 or CDw131 is administered to a swine with SIRS. More particularly, an immune stimulatory amount of an agonist of CD116 or CDw131 is administered to a swine with SIRS.

Another aspect of the invention is a method of treating porcine epidemic abortion and respiratory syndrome (PEARS). An agonist of CD114 (Granulocyte Colony Stimulating Factor Receptor (G-CSFR)) is administered to a swine with PEARS. More particularly, an immune stimulatory amount of an agonist of CD114 (Granulocyte Colony Stimulating Factor Receptor (G-CSFR)) is administered to a swine with PEARS.

Another aspect of the invention is a method of treating PEARS. An agonist of CD116 or CDw131 is administered to a swine with PEARS. More particularly, an immune stimulatory amount of an agonist of CD116 or CDw131 is administered to a swine with PEARS.

The present invention thus opens a new realm of treatment modalities for inflammatory respiratory disease syndromes, both in patients and in animals.

### DETAILED DESCRIPTION OF THE INVENTION

It is a discovery of the present inventors that immune modulatory factors which act at CD114, CD116, and or CDw131 can be successfully used to treat various forms of inflammatory respiratory disease. These include but are not limited to ARDS, IRDS, SARS, PRRS, PEARS, and SIRS.

The immune modulatory factor can be any factor which binds to CD114, CDw131, or CD116, including but not limited to G-CSF, GM-CSF, IL-3, IL-5, and peptidomimetics or non-peptidomimetics of these factors which induce tyrosine phosphorylation of multiple signaling proteins, which stimulate primary bone marrow cells to form granulocytic colonies in vitro, and/or which elevate peripheral blood neutrophil counts. Nartograstim, myelopoietins, circularly permuted G-CSF sequences, SB247464 are among the known mimetics of G-CSF. See, McWherter et al., Biochemistry 14:4564-71, 1999; Feng et al., Biochemistry 14:4553-63, 1999; Tian et al., Science 281:257-59, 1998; and Kuwabara et al., Am. J. Physiology 271:E73-84, 1996. M-CSF may also be used in accordance with the present invention.

The immune modulatory factors are typically growth factors or colony stimulating factors which affect the growth of hematopoietic cells, particularly myeloid cells, including polymorphonuclear leukocytes, monocytes, and macrophages. Such factors include but are not limited to myeloid cell stimulatory factors, polymorphonuclear leukocyte stimulatory factors, and granulocytic cell stimulatory factors. Particularly useful factors are G-CSF, GM-CSF, and M-CSF.

Any form of such factors known in the art can be used. The form may be an isoform or a differently post-translationally modified form of the factor. The factor may be one which is isolated from humans or other primates or mammals. The factor may be one which is made in a recombinant organism, from bacteria to yeast to sheep.

A derivative of the immune modulatory factors of this invention can also be utilized. A derivative includes all modifications to the factor which substantially preserve the functions disclosed herein and include additional structure and attendant function (e.g., PEGylated factors which may exhibit a greater half-life), fusion polypeptides which confer targeting specificity or an additional activity.

Methodologies for preparing derivatives of factors are well known in the art.

The immune modulator factor may be administered both systemically and locally by means that are known in the art. Typically, this will be by subcutaneous injection or intravenous infusion, however other methods such as oral, intraperitoneal, subdermal, and intramuscular administrations can be used. In addition, the factor may be administered with aerosolized delivery, including direct aerosolized delivery.

The immune modulatory factor may also be expressed in vivo, which is often referred to as “gene therapy.” Thus, for example, cells may be engineered with a polynucleotide (DNA or RNA) encoding for the agonist ex vivo, the engineered cells may then be provided to a patient to be treated with the agonist. Such methods are well-known in the art. For example, cells may be engineered by procedures known in the art by use of a retroviral particle containing RNA encoding for the immune modulatory factor.

Local delivery of the immune modulatory factor using gene therapy may provide the factor to the target area (e.g., respiratory tract and more particularly, the lungs).

Doses which are delivered may be the same as those which are delivered to stimulate an immune response in humans for other disease purposes. Typically doses of the factors will be between about 0.1 and 100 μg/kg of body weight per day. More preferably this will be between about 1.0 and 10 μg/kg of body weight per day. Most preferably the dose will be between about 2 and 8 μg/kg of body weight per day.

The determination of an immune stimulatory amount of factor is well within the capability of those skilled in the art. An immune stimulatory amount of a factor refers to that amount of factor that activates acquired immune responses or acquired host defenses, including but not limited to the stimulation of dendritic cells and/or macrophages. Typical dose amounts required to activate an acquired immune response or acquired host defenses are between at least 25 and 350 μg total dose per day, more preferred the typical dose is between at least 50 and 300 μg total dose per day, still more preferred the typical dose is between 100 and 250 μg total dose per day. The dose amount of factor, namely, 50-350 μg total dose, can also be administered with lower frequency (e.g., every other day or 2-3 times per week).

An immune stimulatory amount of a factor can also refer to the amount of factor that activates innate immune cell types. Typical dose amounts required to activate innate immune cells types are greater than 350 μg total dose per day, more preferred greater than 500 μg total dose per day, still more preferred more than 700 μg total dose per day and most preferred more than 1000 μg total dose per day.

Corresponding amounts of peptidomimetics and non-peptidomimetics to achieve the same activity can be used. White blood cell counts can be monitored to maintain a value in the range of 5K and 60K cells/ul. Other cell types expressing these receptors can also be measured including dendritic cells, neutrophils, monocytes, macrophages, and eosinophils. Measured increases vary dependent on the assay and individual, but all cell types increase in response to receptor engagement.

The immune modulatory factor may be used alone or in combination with additional therapies and/or compounds known to those skilled in the art in the treatment of inflammatory respiratory diseases and related disorders. Alternatively, the methods and compounds described herein may be used, partially or completely, in combination therapy.

The immune modulatory factors may also be administered in combination with other known biologic and small molecule therapies for the treatment of inflammatory respiratory diseases, including, for example, but not limited to infleximab, IL-2, IFN-beta-1, IFN-beta-2, etc. Such therapies may be administered prior to, concurrently with or following administration of the immune modulatory factors described herein.

Diseases which are amenable to treatment as described herein include all within the umbrella of inflammatory respiratory disease. Treatment of inflammatory respiratory disease as described herein refers to prevention as well as treatment during initial development of the disease and after disease onset.

The exact dosage of immune modulatory factor will be determined by the practitioner, in light of factors related to the subject that requires treatment. Exact dosage and administration are adjusted to provide sufficient levels of the immune modulatory factor or to maintain or obtain the desired effect. Factor which can be taken into account include the severity of the disease state, general health of the subject, age, weight and gender of the subject, diet, time and frequency of administration, drug combination(s), reaction sensitivities, and tolerance/response to therapy.

One goal of treatment is the amelioration, either partial or complete, either temporary or permanent, of patient symptoms, including reduction of inflammation of the respiratory tract, e.g., improvement in lung tissue swelling; extra-respiratory manifestations of the disease; or epithelial damage. Amelioration can be measured by any method, either through lab analysis or in the clinical setting, such as for example, X-ray analysis of lung tissue swelling, examination of exercise tolerance and/or a patient's requirement for oxygen or ventalatory support. Any amelioration is considered successful treatment. This is especially true as amelioration of some magnitude may allow reduction of other medical treatment which may be more toxic or invasive to the patient.

The present invention is based on the theory that respiratory syndromes result from an immune deficiency, which can be caused by a number of different etiologies. This deficiency provokes a broader compensatory response, amplifying inflammation, activating lymphocytes, and culminating in lung failure.

The GM-CSF receptor is composed of two subunits:
1) Hs.182378 colony stimulating factor 2 receptor, alpha, low-affinity (granulocyte-macrophage) CSF2RA (CD116). CD116 is the GM-CSF receptor alpha chain; the primary binding subunit of the GM-CSF receptor.

CD116 is a Type I transmembrane protein with about 400 amino acids. Extracellular, transmembrane and cytoplasmic domains consist of 297, 27, and 54 amino acid residues, respectively. There is one unit of class I cytokine receptor motif in the extracellular domain and no intrinsic enzymatic activity in the cytoplasmic domain. A number of isoforms are generated by alternative splicing of several soluble forms. All the isoforms are relatively minor species and their physiological function if any is not known. One is a soluble form without the transmembrane domain and the second form is identical to the original one except that the last 25 amino acids of the original receptor is substituted by a 35 amino acids segment.

CD116 binds GM-CSF with low affinity and binds it with high affinity when it is co-expressed with the common beta subunit CDw131 (the common beta subunit (CDw131) of the GM-CSF, IL-3, and IL-5 receptors). Expression of this subunit is found in various myeloid cells including macrophages, neutrophils, eosinophils, dendritic cells and their precursors.

Tavernier et al. (1991) demonstrated that the high affinity receptor for interleukin-5 (IL5R; 147851) and the receptor for granulocyte-macrophage CSF (CSF2R; 306250) share a beta chain. The finding provides a molecular basis for the observation that IL5 (147850) and CSF2 (138960) can partially interfere with each others binding and have highly overlapping biologic activities on eosinophils. Kitamura et al. (1991) demonstrated that the receptor for interleukin-3 (IL3RA; 308385) likewise shares a beta subunit with CSF2R.
2) Hs.265262 colony stimulating factor 2 receptor, beta, low-affinity (granulocyte-macrophage) CSF2RB* (CDw131).

Alternate names for CDw131 are common beta subunit INTERLEUKIN 5 RECEPTOR, BETA; IL5RB INTERLEUKIN 3 RECEPTOR, BETA; IL3RB *138981 GRANULOCYTE-MACROPHAGE COLONY-STIMULATING FACTOR RECEPTOR, BETA; CSF2RB

CDw131 does not bind any cytokine by itself. However, it is a component of the high affinity IL-3, GM-CSF and IL-5 receptors. CDw131 is tyrosine phosphorylated upon binding of these cytokines to the high affinity receptors. JAK2 tyrosine kinase is associated with CDw131 and tyrosine phosphorylates upon stimulation. Tyrosine phosphorylated CD131 binds various signaling molecules with an SH2 domain. These include Shc, Grb2, SHP1, SHP2, P13 kinase and STAT5, making it a key signal transducing molecule of the IL-3, GM-CSF and IL-5 receptors.

All patents and patent applications and all references to journal articles, etc. cited in this disclosure are expressly incorporated herein by reference. The above disclosure generally describes the present invention. Additional information concerning the invention can be obtained by reference to the examples below which are provided for purposes of illustration only and are not intended to limit the scope of the invention.

### EXAMPLES

### Example 1

This example shows the protocol for a study of the method of the present invention using GM-CSF for the treatment of SARS patients.

Study Design:

Phase II, open label, non-controlled multi-center trial

Patient Population:
Presumed, probable, or established diagnosis of SARS
Pulmonary complications requiring mechanical ventilation
Acute onset of illness with:
a) PaO₂/FiO₂≦300 (ALI) or PaO₂/FiO_{2≦200} (ARDS)
b) Bilateral infiltrates consistent with pulmonary edema on frontal chest radiograph. The infiltrates may be patchy, diffuse, homogeneous, or asymmetric.
c) Requirement for positive pressure ventilation via an endotracheal tube.
d) No clinical evidence of left atrial hypertension. If measured, pulmonary arterial wedge pressure≦18 mm Hg.
e) Criteria a-c must occur together within a 24-hour interval.
Exclusion criteria
a) Age<18 years
b)>7 days elapsed following institution of mechanical ventilation
c) Pregnancy
d) Chronic respiratory failure
e) Left ventricular failure
f) Neutropenia (absolute neutrophil count<1000 cell/mm³)
g) History of hematological malignancy or bone marrow transplantation
h) Entry in other intervention clinical trials
i) Decision of the patient or attending physicians to forego aggressive care
j) Informed consent

Endpoints:
Duration of mechanical ventilation
Clinical recovery
Time in the hospital; time in intensive care unit

Treatment Schedule:

Slow intravenous over 4-5 hours of GM-CSF at 250 μg/m²/day for 14 days, equal to roughly 6-7 μg/kg/day in a 70 kg individual.

GM-CSF may be administered through either central venous access or a peripheral Intravenous line.

### Example 2

This example shows the schedule for the treatment of swine with respiratory disease. GM-CSF is injected subcutaneously at 10 μg/kg/day for 14 days. If necessary, the dose is adjusted.

What is claimed is:

1. A method of treating inflammatory respiratory disease comprising:
administering to a patient or animal with inflammatory respiratory disease an agonist of CD114.

2. A method of treating inflammatory respiratory disease comprising:
administering to a patient or animal with inflammatory respiratory disease an agonist of CD116 and/or CDw131.

3. The method of claim 1 or 2 wherein the patient has severe acute respiratory disease (SARS).

4. The method of claim 1 or 2 wherein the inflammatory respiratory disease is selected from the group consisting of: adult (acute) respiratory disease syndrome (ARDS).

5. The method of claim 1 or 2 for the treatment of animals with inflammatory respiratory disease.

6. The method of claim 1 or 2 wherein the disease is selected from the group consisting of porcine reproductive and respiratory syndrome (PRRS).

7. The method of claim 1 or 2 wherein the disease is swine infertility and respiratory syndrome (SIRS).

8. The method of claim 1 or 2 wherein the disease is porcine epidemic abortion and respiratory syndrome (PEARS).

9. The method of claims 1-8 wherein the amount of colony stimulating factor administered reduces the symptoms.

10. The method of claims 1-8 wherein the amount of colony stimulating factor administered induces remission.

11. The method of claim 1 wherein the agonist is G-CSF.

12. The method of claim 2 wherein the agonist is GM-CSF.

13. The method of claim 2 wherein the agonist is sargramostim.

14. The method of claim 1 wherein the agonist is pegylated G-CSF.

15. The method of claim 2 wherein the agonist is pegylated GM-CSF.

16. The method of claim 2 wherein the agonist is pegylated sargramostim.

17. The method of claim 1 or 2 wherein the agonist is administered in a slow-release formulation.
